(19) United States
(12) Patent Application Publication
(10) Pub. No.: US 2003/0072714 A1
(43) Pub. Date: Apr. 17, 2003

(54) Microfluidized leishmania lysate and methods of making and using thereof

(76) Inventors: Alan J. Magill, Kensington, MD (US); John M. Stiteler, Springfield, VA (US); Max Grogl, Columbia, MD (US); Edgar D. Rowton, College Park, MD (US); Kenneth H. Eckels, College Park, MD (US); William R. Ballou, Silver Spring, MD (US)

Correspondence Address: Office of the Staff Judge Advocate, U.S. Army Medical Research and Materiel Command, ATTN: MCMR-JA (Ms. Elizabeth Arwine), 504 Scott Street, Fort Detrick, MD, 21702-5012, US

(21) Appl. No.: 09/975,020

(22) Filed: Oct. 12, 2001

(51) Int. Cl. A61K 49/00; G01N 33/53; G01N 33/569

(52) U.S. Cl. 424/9.81; 435/7.22

(57) ABSTRACT

Disclosed herein are microfluidized lysate preparations of Leishmania parasites and methods of making thereof. Also disclosed are methods of using the microfluidized lysate preparations in skin test antigen assays as well as kits comprising the microfluidized lysate preparations. The microfluidized lysate preparations are made under current good manufacturing practice and may therefore be standardized and such preparations may be produced with consistently.

### ACKNOWLEDGMENT OF GOVERNMENT SUPPORT

This invention was made by employees of the United States Army. The government has rights in the invention.

### BACKGROUND OF THE INVENTION

1. Field of the Invention

This invention relates generally to microfluidized Leishmania lysate preparations. In particular, the present invention relates to microfluidized Leishmania lysate preparations for assays and immunogenic compositions.

2. Description of the Related Art

Leishmaniasis is a serious and sometimes fatal disease. The World Health Organization (WHO) reports that about 40 million individuals are infected with Leishmania in 88 countries. Transmission of leishmaniasis is especially problematic in Mediterranean Africa, Asia, and Latin America. Leishmaniasis is a threat individuals who travel to or live in endemic areas. There are about 10 to about 25 new cases of cutaneous leishmaniasis in U.S. military personnel each year. There have been some notable outbreaks of leishmaniasis which yielded about 50 cases per year and attach rates as high as 50% in military personnel. There is one instance when Canadian Paratroopers suffered an attack rate of over 90% in as few as 6 hours of being in Leishmania “hot spots” in French Guyana. Additionally, in 1991, the Gulf War presented a new clinical syndrome of visceral leishmaniasis caused by L. tropica. See Magill, A. J. et al. (1993) N. Engl. J. Med. 328(19):1383-1387; and Magill, A. J. et al. (1994) Clin. Infect. Dis. 19(4):805-806.

Unfortunately, current acceptable diagnostic practices lack the means for efficiently and accurately identifying those infected or exposed to the disease-causing parasite as the majority of Leishmania infections do not result in overt clinical manifestations. See Pampiglione, S. et al. (1974) Trans. Roy. Soc. Trop. Med. Hyg. 68(6):447-453; Ho, M. et al. (1982) Trans. Roy. Soc. Trop. Med. Hyg. 76(6):741-746; and Evans, T. et al. (1992) J. Infect. Dis. 166:1124-1132. There are many assays designed to detect a cell-mediated immune response in exposed individuals. However, these test are costly, time consuming and hard to perform. In addition, prior art methods for detecting cell-mediated immunity against leishmanial antigens, such as T-cell proliferation and cytokine production, are not only difficult to standardize, but they require the collection and cyropreservation of peripheral blood mononuclear cells. For these reasons present assays design to detect cell-mediated responses are not technically or logistically practical for screening large numbers of individuals.

Serology, primary ELISA, IFA and agglutination assays are somewhat useful in the diagnosis of visceral leishmaniasis. Nevertheless, these assays are of little use for diagnosing cutaneous and mucocutaneous leishmaniasis where the antibody titers are low. As a result, the prevention of leishmanial epidemics is greatly hindered and patient management is difficult.

Although there are many prior art methods for detecting cell-mediated immunity against leishmanial antigens, such as T-cell proliferation and cytokine production, the prior art methods are not only difficult to standardize, but they require the collection and cyropreservation of peripheral blood mononuclear cells and are not technically or logistically practical for screening large numbers of individuals.

Skin test assays are a practical way to screen large numbers of individuals as delayed hypersensitivity occurs with most individuals having cutaneous, mucocutaneous, post kala-azar dermal, and cured visceral leishmaniasis may be measured by the leishmanin test. The use of crude leishmanial antigens to elicit DTH was first reported by Montenegro in 1926. See Montenegro, J. (1926) Archives Dermatology and Syphilology 13187-194. Since then, many different leishmanial preparations for skin tests have been prepared and used in endemic areas. Most employ a locally acquired strain of Leishmania and make a crude antigenic preparation comprising either whole promastigotes or disrupted promastigotes (sonicated or freeze-thawed) or soluble antigens.

These leishmanial preparations are problematic as the preparations suffer from lack of standardization, undefined sensitivity and specificity, unknown sensitizing capacity, and unknown dose or response relationships between antigen content, clinical syndrome, or infecting parasite. Further, the prior art preparations have short-term shelf life. For example, Reed et al. discloses a leishmanial skin test antigen that is safe and effective. See Reed, S. et al. (1986) Am. J. Trop. Med. Hyg. 35:79-85. SDS-PAGE of a fresh preparation provided a series of distinct bands; however, SDS-PAGE of an older stored batch did not provide distinct bands thereby indicating protein degradation by proteases. Other problems of leishmanin tests include the lack of a universal and standardized Leishmania skin test antigen which may be used to set the standard of care or diagnosis in various countries. These problems of the prior art preparations and methods have prevented development and approval of a leishmanial skin test antigen for clinical use.

In summary, the strengths of the leishmanin tests include delayed hypersensitivity as an important feature of all forms of leishmaniasis that may be measured and that the parasitic antigens elicit a cutaneous delayed-type hypersensitivity response in most individuals with cutaneous leishmaniasis, mucocutaneous leishmaniasis, post kala-azar dermal leishmaniasis, and cured visceral leishmaniasis. The shortcomings of the leishmanin tests include false positive reactions that can be high in areas where there is a background of leishmaniasis, as many individuals in the healthy populations of endemic leishmaniasis areas having no evidence of past infection may show high rates of leishmanin sensitivity. The shortcomings also include cross-reactions with cases of glandular tuberculosis and lepromatous leprosy, cross-reactivity between Leishmania strains as heterologous antigens often give smaller reactions, lack of a universal and standardized Leishmania skin test antigen, unknown sensitizing capacity, unknown stability, unknown dose/response relationships between antigen content and clinical syndrome or infecting parasite, and the standard of care of using Leishmania skin test antigen in many endemic countries is not approved by drug approval agencies such as the U.S. Food and Drug Administration.

Thus, a need still exists for an effective, convenient, simple, and cost effective assay to detect cell-mediated immunity against leishmanial antigens that renders itself to be manufactured following cGMP so it can be approved by the FDA.

### SUMMARY OF THE INVENTION

The present invention generally relates to microfluidized leishmanial antigens and methods of making and using thereof.

In some embodiments, the present invention relates to a method of preparing a microfluidized lysate preparation comprising microfluidizing a slurry of at least one Leishmania parasite through a chamber and disrupting the leishmania parasite with a sudden release of pressure. The method further comprises heat treating the microfluidized lysate preparation. The Leishmania parasite may be L. tropica, L. mexicana, L. guyanensis, L. braziliensis, L. major, L. donovani, L. chagasi, L. amazonensis, L. peruviana, L. panamensis, L. pifanoi, L. infantum, or L. aethiopica.

In some embodiments, the present invention relates to a microfluidized lysate preparation made by microfluidizing a slurry of at least one Leishmania parasite through a chamber and disrupting the leishmania parasite with a sudden release of pressure and heat treating the microfluidized lysate preparation. The Leishmania parasite may be L. tropica, L. mexicana, L. guyanensis, L. braziliensis, L. major, L. donovani, L. chagasi, L. amazonensis, L. peruviana, L. panamensis, L. pifanoi, L. infantum, or L. aethiopica.

In some embodiments, the present invention relates to a skin test antigen assay for detecting whether a subject has been exposed to a Leishmania parasite or was afflicted with Leishmaniasis comprising administering to the subject an antigenic amount of at least one microfluidized lysate preparation of the present invention and observing any immunogenic response to the microfluidized lysate preparation. The Leishmania parasite may be L. tropica, L. mexicana, L. guyanensis, L. braziliensis, L. major, L. donovani, L. chagasi, L. amazonensis, L. peruviana, L. panamensis, L. pifanoi, L. infantum, or L. aethiopica. An immunogenic response indicates that the subject has been exposed to a Leishmania parasite or was afflicted with Leishmaniasis. Preferably, an induration of about 5 mm or greater observed indicates that the subject has been exposed to a Leishmania parasite or was afflicted with Leishmaniasis. In preferred embodiments, the antigenic amount of the microfluidized lysate preparation comprises about 5 μg to about 30 μg of total protein. The microfluidized lysate preparation is administered intradermally to the volar surface of the forearm of the subject.

In some embodiments, the present invention relates to a kit comprising the microfluidized lysate preparation of the present invention and directions for determining whether a subject has been exposed to a Leishmania parasite or was afflicted with Leishmaniasis. The Leishmania parasite may be L. tropica, L. mexicana, L. guyanensis, L. braziliensis, L. major, L. donovani, L. chagasi, L. amazonensis, L. peruviana, L. panamensis, L. pifanoi, L. infantum, or L. aethiopica. The kit may further comprise at least one pharmaceutical for treating systemic anaphylaxis such as epinephrine, diphenhydramine, and methyl prednisolone. The kit may further comprise at least one pharmaceutical for treating local reactions to the microfluidized lysate preparation such as hydrocortisone, hydrocortisone cream, acetaminophen, or diphenhydramine.

In some embodiments, the present invention relates to antibodies raised against the microfluidized lysate preparation of the present invention.

In some embodiments, the present invention relates to a method of determining whether a subject has been exposed to a given Leishmania parasite comprising administering to the subject a panel of antigenic compositions comprising a plurality of microfluidized lysate preparations prepared from a plurality of Leishmania parasites and detecting a presence of an immunogenic reaction that is characteristic to exposure to the given Leishmania parasite. The plurality of Leishmania parasites may include at least one parasite belonging to the group consisting of L. tropica, L. mexicana, L. guyanensis, L. braziliensis, L. major, L. donovani, L. chagasi, L. amazonensis, L. peruviana, L. panamensis, L. pifanoi, L. infantum, and L. aethiopica.

In some embodiments, the present invention relates to a method of immunizing a subject against Leishmaniasis comprising administering to the subject an immunogenic amount of the microfluidized lysate preparation.

In some embodiments, the present invention relates to a pharmaceutical composition comprising the microfluidized lysate preparation and a pharmaceutically acceptable stabilizer such as phenol. In preferred embodiments, the pharmaceutical composition is in the form of a liquid which may be frozen or freeze-dried.

In some embodiments, the present invention relates to a method for determining post infection of cutaneous leishmaniasis, mucocutaneous leishmaniasis, or post-kala-azar dermal leishmaniasis in a subject comprising administering to the subject an antigenic amount of at least one microfluidized lysate preparation and observing any immunogenic response to the microfluidized lysate preparation.

In some embodiments, the present invention relates to a method for epidemiologically diagnosing cutaneous leishmaniasis, mucocutaneous leishmaniasis, or post-kala-azar dermal leishmaniasis in a subject comprising administering to the subject an antigenic amount of at least one microfluidized lysate preparation and observing any immunogenic response to the microfluidized lysate preparation.

In some embodiments, the present invention relates to a method for determining the pattern of present and past leishmaniasis in a subject comprising administering to the subject an antigenic amount of at least one microfluidized lysate preparation and observing any immunogenic response to the microfluidized lysate preparation.

It is to be understood that both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention and together with the description serve to explain the principles of the invention.

### DESCRIPTION OF THE DRAWINGS

This invention is further understood by reference to the drawings wherein:

FIG. 1 is a flow diagram showing the process for making a Leishmaniasis microfluidized lysate according to the present invention.

### DETAILED DESCRIPTION OF THE INVENTION

The present invention allows the detection of post infections and epidemiological diagnosis of all forms of leishmaniasis. The present invention may be used for screening of individuals such as U.S. Service Members who may have been exposed to Leishmania parasites after deployment to Leishmaniasis endemic areas. The present invention may be used in the clinical diagnosis of cutaneous leishmaniasis, mucocutaneous leishmaniasis, and post-kala-azar dermal leishmaniasis in subjects such as U.S. Service Members. The present invention may be used to determine the pattern of past and present infections of leishmaniasis.

The present invention provides microfluidized lysate preparations comprising at least one antigen from at least one Leishmania parasite and methods of making and using thereof. The microfluidized lysate preparations of the present invention may be used to elicit induration that is consistent with delayed type hypersensitivity (DTH) in an individual previously infected with or exposed to at least one Leishmania parasite. Preferably, the microfluidized lysate preparation is injected intradermally. Thus, the present invention also provides a skin test assay for detecting whether a subject has been exposed to a Leishmania parasite or is or has been afflicted with Leishmaniasis comprising at least one microfluidized lysate preparation of at least one Leishmania parasite.

As described herein, the microfluidized lysate of the present invention comprises at least one solubilized antigen of at least one Leishmania parasite. The microfluidized lysate preparation may further comprise a mixture of amino acids, lipids, and carbohydrates. The Leishmania parasite may be specifically selected for a particular reason. For example, an Old World parasite, such as L. tropica strain WR1063, may be used in order to detect L. tropica infections acquired in Southwest Asia. However, any Leishmania parasite, Old World or New World, may be selected by one of ordinary skill in the art, prepared, and used according to the present invention. Examples of a few Leishmania parasites include L. tropica, L. mexicana, L. guyanensis, L. braziliensis, L. major, L. donovani, L. chagasi, L. amazonensis, L. peruviana, L. panamensis, L. pifanoi, L. infantum, L. aethiopica.

The L. tropica strain WR1063 used herein was cloned by mechanical-single cell isolation and given the designation, CL1. The microfluidized lysate preparations of the present invention were prepared under current good manufacturing practices (cGMP). Disruption of the promatigotes was accomplished by microfluidization comprising passing a parasite slurry through a chamber and disrupting the cells by the sudden release of pressure. Not only does microfluidization allow the release of membrane embedded antigens, but it provides a reproducible method for providing large volumes of preparations. Although the potency of the antigens in the microfluidized lysate preparations were not affected by prolonged storage at about 2° C. to about 8° C., the microfluidized lysate preparations were heat-treated at about 95° C. in a water bath for about 30 minutes to deactivate the proteolytic enzymes.

Specifically, a strain of vicerotropic leishmania, WR1063, was isolated, cloned, and then characterized as L. tropica by isoenzyme analysis. Other Leishmania parasites may be used such as L. mexicana as described in Example 2. A master seed lot and a production seed lot of the clone were used to initiate individual bulk production lots of promastigotes. Three bulk lots (about 1×10¹¹ promastigotes each) were pooled in a vial and immediately placed on dry ice. The pooled promastigotes were then thawed by placing the vial in a water bath at about 56±2° C. Immediately upon thawing, the vial was placed at about 4±2° C. to cool. After cooling, the contents of the vial were transferred to a pre-tared 250 ml sterile centrifuge bottle on ice using a 10 cc sterile syringe. Two 0.5 ml samples of the suspended cells were pipetted and placed in a sterile 1.5 ml Nunc cryovial (Fisher Scientific, Pittsburgh, Pa.) and stored at about −80±10° C.

A microfluidizer, Model #M-110S was used to extract the L. tropica soluble proteins. The regulator was set and wet ice was put into the cooling jacket of the microfluidizer. The pump was primed by placing the inlet tubing into 500 ml of 0.001% Tween 80 and 0.9% saline and then opening the air valve. With the regulator at about 100±5 psi, the inlet tubing was inserted into the promastigotes suspension and run through the microfluidizer. Because the cracking pressure fluctuates, an average reading was taken. Cracked cells were collected into the reservoir containing the uncracked cells and cracking continued for about 10±1 minutes. The pre- and post-cracking temperatures were recorded. The 250 ml tube containing the lysed promastigotes was capped and stored at about 4±2° C.

The cracked cells were dispensed into a sterile 250 ml centrifuge bottle and centrifuged at about 3,100±200 rpm (1566×g) in a Sorvall GSA rotor within a RC-5 Sorvall centrifuge at a time setting of about 30±1 minutes and at a temperature of about 4±2° C. The bottle was removed and placed in a Class II Biohazard cabinet. The supernatant was poured of into a second sterilized 350 ml centrifuge tube and stored at about 4±2° C. The pellet was then suspended with about 20 ml of Buffer B comprising, 0.001% Tween 80 diluted with 0.9% saline, and vortexed with vortex mixer. Ice was added into the cooling jacket of the microfluidizer as needed. When the regulator was at about 100±5 psi, the inlet tubing was inserted into the suspension and run through the microfluidizer in a continual fashion for about 10 minutes. The cracked cells were collected into a 250 ml centrifuge bottle containing the lysed promastigotes and placed on ice. The pre- and post-cracking temperatures were recorded. Then two 0.5 ml of the cracked cell samples were taken and stored at about −80±10° C. On ice, the cracked cells and the supernatant were combined and mixed by swirling in the container.

To avoid any possibility of breakdown, the post-cracked cells were immediately centrifuged at about 12,200±200 rpm (about 23,435 to about 25,062×g) in a Sorval GSA rotor within a RC-5 Sorvall centrifuge for about 30±1 minutes at about 4±2° C. Then the bottle was removed and placed in BSC, the supernatant was poured off into a sterile 250 ml graduated cylinder and the pellet in the 250 ml bottle was retained.

A biosafety cabinet was sterilized as well as other items placed in the cabinet with 70% alcohol. A UV lamp was kept on in the cabinet for 15 minutes prior to use. The bulk lysate and centrifuged promastigotes were filtered. The volume of centrifuged promastigotes solution was estimated and the in process purified bulk lysate was aseptically filtered using a 500 ml 0.22 μm Millipore filtration unit (Fisher Scientific, Pittsburgh, Pa.). The filtered bulk was aseptically transferred into a preweighed, sterile bottle. The bottle of the bulk was reweighed and the bulk weight was determined. Two 0.5 ml aliquots were taken aseptically and retained at about −80±10° C. One 1.0 ml sample was taken for protein concentration testing by the Bio-Rad method (Bio-Rad Laboratories, Hercules, Calif.) and for SDS-PAGE.

The bulk clarified lysate was tightly sealed and stored at about 4±2° C. until treated with heat. 25% glycerol was added to the bulk clarified lysate to give a final concentration of about 1% and then mixed by swirling. Then the total weight was determined. Then the bulk clarified lysate was incubated for about 30±2 minutes using a water bath at about 93±2° C. After heat treatment, the bulk was cooled at about 4±2° C. Aseptically two 0.5 ml aliquots were taken and retained at about −80±10° C. One 1.0 ml sample was taken for protein concentration testing by the Bio-Rad method (Bio-Rad Laboratories, Hercules, Calif.) and for SDS-PAGE.

The final clarified lysate protein concentration was aseptically adjusted to about 0.35±0.05 mg/ml with about 0.4% phenol buffer. Then samples were aseptically obtained and assayed for protein content by BCA, endotoxin content LAL (gel-clot), pH, sterility, HPLC, purity by SDS-PAGE, color and appearance.

An Omnispense dispensing pump (Wheaton Science Products, Millvilee, N.J.) was used to dispense about 1.0 g±5% of the final clarified lysate into 10 ml depyrogenized, sterile, glass vials. The bulk final clarified lysate was swirled on an orbital shaker at about 60 rpm during the filling operation to ensure homogeneity. The final vials were inspected and weight checked. The vials were labeled and tested for sterility, pH, protein concentration, LAL, SDS-PAGE, HPLC and immunogenicity.

The resulting microfluidized lysate preparation was used in a skin test assay for detecting whether a subject had been exposed to a Leishmania parasite, such as L. tropica. Clearly, it is well within the ability of one of ordinary skill in the art to produce microfluidized lysate preparations from other Leishmania parasites, such as L. mexicana and L. guyanensis, according to the method disclosed herein in order to determine whether a subject has been infected with or exposed to other Leishmania parasites. Additionally, one of ordinary skill in the art may prepare a microfluidized preparation from more than one Leishmania parasite. Thus, the present invention also provides a multivalent microfluidized lysate preparation prepared from at least two different Leishmania parasites. For example, the preparation may comprise the microfluidized lysate of L. tropical L. mexicana, and L. guyanensis.

The skin test assay of the present invention comprises administering to a subject an antigenic amount of a microfluidized lysate of the present invention. Preferably, the subject is a mammal, more preferably, the subject is human. Preferably, the microfluidized lysate preparation is administered intradermally, more preferably, the microfluidized lysate preparation is injected into the volar surface of the forearm of the subject. Induration is then measured. Preferably, induration is measured at least once at about 24 to about 72 hours after the microfluidized lysate preparation was administered. More preferably, induration is measured at about 48 hours after induration as true delayed type hypersensitivity responses in humans are maximal at about 48 hours. As used herein, an “antigenic amount” is an amount which provides a positive induration response in a subject known to be exposed to a leishmanial parasite. Antigenic amounts range from about 0.01 to about 1.0 ml, preferably about 0.05 to about 0.5 ml, more preferably about 0.1 ml. A positive response indicates previous exposure and sensitization to the antigen has occurred and is recalled, and an antigen specific inflammatory response takes place, thereby indicating that the subject was infected with or had been exposed to at least one Leishmania parasite. A positive response in humans is an induration of about 5 mm or more.

As explained in Example 3, the microfluidized lysate preparations of the present invention are safe and immunogenic. Therefore, the present invention also provides immunogenic compositions and vaccines comprising the microfluidized preparations of the present invention. The vaccine or immunogenic compositions of the present invention may be used in combination with an adjuvant, a pharmaceutically acceptable excipient, or both. Thus, the present invention also provides methods for immunizing subjects against diseases, infections, or disorders associated with Leishmania parasites, such as Leishmaniasis, all forms of leishmaniases, and diseases as Chagas Disease, and African trypanosomiasis were common crossreacting antigens with immunoprotective properties.

Since the method of preparing the microfluidized lysate preparations as described herein may be standardized and reproduced to prepare batches of microfluidized lysate preparations having substantially similar characteristics, such as potency and specificity, the microfluidized lysate preparations may be used to purify or screen for ligands which bind to leishmanial antigens or antibodies raised against the microfluidized lysate of the present invention with consistency.

Additionally, the microfluidized lysate preparations of the present invention are suitable for use as immunogens to raise anti-leishmanial antibodies. The antibodies may be prepared by immunizing a suitable subject, e.g., rabbit, goat, mouse or other mammal, with the microfluidized lysate of the present invention by methods standard in the art. The microfluidized lysate preparation may further include an adjuvant, such as Freund's complete or incomplete adjuvant, or a similar immunostimulatory agent. Accordingly, the present invention also provides antibody preparations made by immunizing a suitable subject with a microfluidized lysate preparation of the present invention. The antibodies produced by the subject may be isolated or purified by methods standard in the art.

The antibodies raised against the microfluidized lysate of the present invention may be used to isolate leishmanial antigens by methods standard in the art, such as affinity chromatography or immunoprecipitation. The antibodies raised against the microfluidized lysate of the present invention may be used to diagnostically to monitor protein levels in tissue as part of a clinical testing procedure, e.g., to determine the efficacy of a given treatment regimen. Detection can be facilitated by coupling the antibody to a detectable label or marker. Examples of detectable labels and markers include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ²⁵¹I, ¹³¹I, ³⁵S or ³H.

The microfluidized lysate preparation, antibodies, or antigens of the present invention may be incorporated into a pharmaceutical composition suitable for administration. Such compositions typically comprise the microfluidized lysate preparation, antibodies, or antigens of the present invention and a pharmaceutically acceptable carrier. Preferably, pharmaceutical compositions of the present invention comprise an antigenic amount of the microfluidized lysate preparation or antigen or a therapeutically effective amount of antibodies raised against the microfluidized lysate preparation, and an inert, pharmaceutically acceptable carrier or diluent. As used herein, “pharmaceutically acceptable carrier” includes any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Preferably, the pharmaceutical compositions of the present invention include sterile saline comprising 0.4% phenol.

The antigenic activity of the microfluidized lysate of the present invention may be measured by any of the methods available to those skilled in the art, including in vitro and in vivo assays. Examples of suitable assays for activity measurements are provided herein. Properties of the microfluidized lysate, such as protein content, endotoxin content, pH, sterility, purity, and color, may be assessed, for example, by methods standard in the art. Other pharmacological methods may also be used to determine the efficacy of the microfluidized lysate as antigenic compositions.

The following examples are intended to illustrate but not to limit the invention.

### EXAMPLE 1

### Process for Making L. guyanensis Microfluidized Lysate

Source material and production of a research seed for the L. guyanensis Leishmania Skin Test, LSTA-Lg (BPR-2334-RS) was conducted as follows. A parasite specimen was obtained from an active dermal lesion from a human subject otherwise healthy with unremarkable medical history. The subject tested negative in screening with viral/STD panel (HIV, HTLV, CMV, HCV, HBV and syphilis). The subject's travel history was determined.

The absence of adventitious agents in all media components used in manufacturing process was determined and (The Fetal Bovine Serum (FBS) Type II, was mycoplasma and bovine virus tested, screened for bacteriophage, tested for endotoxin, tested for AVA, cell culture tested, heat-inactivated, and from a non BSE country (Gibco Life Technologies, Grand Island, N.Y.; Fetal Bovine Serum, Qualified, Origin-United States, Catalogue Number 26140, Lot Number 1016982). The promastigotes cultured from this specimen, (strain WR2334), were determined by isoenzymes to be L. guyanensis. No known viral contaminants, such as LRV1-4, were detected in the promastigotes. A clonal line, WR2334 Clone D2 (WR2334-D2), was established through serial endpoint dilution and microscopic visualization of a single promastigote. This source seed stock of this clone was then expanded in culture, aliquoted and cryopreserved in liquid nitrogen. Sterility and morphology were determined by conventional methods.

A master cell bank for the L. guyanensis (WR2334-Clone D2) Leishmania Skin Test, LSTA-Lg (BPR-377-00, Lot No. 0705) was manufactured under defined cGMP conditions from cryopreserved aliquots of WR2334-Clone D2, by culturing and expansion to roller bottles in appropriate medium. The cultured promastigotes were harvested by centrifugation, washed, suspended in cryopreservation medium, aliquoted, and stored in liquid nitrogen. Sterility and morphology were determined by conventional methods.

A working cell bank for the L. guyanensis (WR2334-Clone D2) Leishmania Skin Test, LSTA-Lg (BPR-377-00, Lot No. 0705) was manufactured under defined cGMP conditions from cryopreserved aliquots of WR2334-Clone D2 Master Cell Bank, by culturing and expansion to roller bottles in appropriate medium. The cultured promastigotes were harvested by centrifugation, washed, suspended in cryopreservation medium, aliquoted, and stored in liquid nitrogen. Sterility and morphology were determined by conventional methods.

A bulk lot for L. guyanensis (WR2334-Clone D2) Leishmania Skin Test LSTA-Lg (BPR-383-00, Lot No. 0707) was produced. Specifically, bulk promastigotes were cultured in Schneider's Drosophila Medium (SDM), supplemented with 20% fetal bovine serum (heat inactivated). The inoculum was initiated in flasks and when the culture set point was reached, it was transferred to roller bottles and expanded. Promastigotes were harvested by centrifugation when a sufficient quantity was been obtained. The culture was maintained in healthy, log-growth phase throughout the production run, including the time of harvest. Harvested promastigotes were centrifuged and washed 5 times. The pellets were reconstituted in sterile saline USP and manually filled in 5.0 mls aliquots at 109 promastigotes/ml. The vials were labeled, stored and frozen at −80° C. for subsequent production and purification steps. Sterility and morphology were determined by conventional methods.

The bulk lot for L. guyanensis (WR2334-Clone D2) Leishmania Skin Test LSTA-Lg (BPR-389-00, Lot No. 0716) was purified. Specifically, cryopreserved vials were thawed and processed through a microfluidizer (MFL#1). The material was centrifuged at 3,100 rpm for 30 minutes and the supernatant was saved. The pellet was resuspended and microfluidized again (MFL#2). The material was centrifuged at 12,200 rpm for 30 minutes and the two supernatants combined. The pooled supernatants were placed in a 0.22 micron sterile filter unit (500 ml) and filtered. The filtrate was then heat-treated at 90° C. for 15-20 minutes to inactivate parasite proteases. The final filtered and heat-treated bulk was formulated and the protein concentration adjusted to about 0.35 mg per ml. The lot was bottled and stored at 4° C. The bulk material was tested for the following characteristics: Visual Inspection: Color, Appearance; Protein Content: micro-BCA (prior to phenol addition); Purity/Identity: SDS-PAGE, HPLC; and Quality: Sterility; Rabbit Pyrogen, Endotoxin (LAL), pH.

The final product of L. guyanensis (WR2334-Clone D2) Leishmania Skin Test LSTA-Lg (BPR-390-00, Lot No. 0717) was placed in vials. Specifically, the formulated, bulk LSTA-Lg was aseptically filled in 1.0 ml aliquots into 10 ml Type I glass vials, closed with 20 mm rubber closures and stored at 4° C. The samples were tested for the following characteristics: Visual Inspection: Color, Appearance, Homogeneity, and Viscosity; Purity/Identity: SDS-PAGE, HPLC; Quality: Sterility, Rabbit Pyrogenicity, General Safety, Endotoxin (LAL); pH; Potency: In vivo Immunogenicity (Guinea pig DTH); and Stability: Time Zero, 3, 6, 12 months, 1, 2 and 3 years at 4, 37, and −80° C.

### EXAMPLE 2

### Production of Heat-Treated Leishmania Skin Test Injectable

Bulk lots of L. mexicana promastigotes in dry ice were pooled by thawing the bulk lots in a water bath at 56±2° C. Immediately upon thawing, the bulk lots were placed at 4±2° C. to cool. After cooling, the contents of the vial were transferred to a pre-tared 250 ml sterile centrifuge bottle on ice using a sterile 5 ml pipet. Two 0.5 ml samples of the suspended cells were pipetted and placed in a 1.5 ml sterile Nunc cryovial (Fisher Scientific, Pittsburgh, Pa.) and stored at −80±10° C.

A microfluidizer, Model #M-110S was washed and autoclaved per the manufacturer's directions. The regulator was set and wet ice was put into the cooling jacket of the microfluidizer. The pump was primed by placing the inlet tubing into 500 ml of 0.001% Tween 80 and 0.9% saline and then opening the air valve. With the regulator at 100±5 psi, the inlet tubing was inserted into the promastigotes suspension and run through the microfluidizer. Because the cracking pressure fluctuates, an average reading was taken. Cracked cells were collected into the reservoir containing the uncracked cells and cracking continued for 10±1 minutes. The pre- and post-cracking temperatures were recorded. The 250 ml tube containing the lysed promastigotes was capped and stored at 4±2° C.

The cracked cells were dispensed into a sterile 250 ml centrifuge bottle and centrifuged at 3,100±200 rpm (1566×g) in a Sorvall GSA rotor within a RC-5 Sorvall centrifuge at a time setting of 30±1 minutes and at a temperature of 4±2° C. The bottle was removed and placed in a Class II Biohazard cabinet. The supernatant was poured of into a second sterilized 350 ml centrifuge tube and stored at 4±2° C. The pellet was then suspended with 20 ml of Buffer B, comprising 0.001% Tween 80 diluted with 0.9% saline, and vortexed with vortex mixer. Ice was added into the cooling jacket of the microfluidizer as needed. When the regulator was at 100±5 psi, the inlet tubing was inserted into the suspension and run through the microfluidizer in a continual fashion for 10 minutes. Cracked cells were collected into a 250 ml centrifuge bottle containing the lysed promastigotes and placed on ice. The pre- and post-cracking temperatures were recorded. Then two 0.5 ml of the cracked cell samples were taken and stored at −80±10° C. On ice the cracked cells and the supernatant were combined and mixed by swirling in the container.

To avoid any possibility of breakdown, the post-cracked cells were immediately centrifuged at 12,200±200 rpm (23,435-25,062×g) in a Sorval GSA rotor within a RC-5 Sorvall centrifuge for 30±1 minutes at 4±2° C. Then the bottle was removed and placed in BSC, the supernatant was poured off into a sterile 250 ml graduated cylinder and the pellet in the 250 ml bottle was retained.

A biosafety cabinet was sterilized as well as other items placed in the cabinet with 70% alcohol. A UV lamp was kept on in the cabinet for 15 minutes prior to use. The bulk lysate and centrifuged promastigotes were filtered. The volume of centrifuged promastigotes solution was estimated and the in process purified bulk lysate was aseptically filtered using a 500 ml 0.22 μm Millipore filtration unit (Fisher Scientific, Pittsburgh, Pa.). The filtered bulk was aseptically transferred into a preweighed, sterile bottle. The bottle of the bulk was reweighed and the bulk weight was determined. Two 0.5 ml aliquots were aseptically taken and retained at −80±10° C. One 1.0 ml sample was taken for protein concentration testing by the Bio-Rad method (Bio-Rad Laboratories, Hercules, Calif.) and for SDS-PAGE.

The bulk clarified promastigotes lysate was tightly sealed and stored at 4±2° C. until treated with heat. 25% glycerol was added to the bulk clarified promastigotes lysate to give a final concentration of 1% and then mixed by swirling. The total weight of the in process bulk was determined. Then the bulk clarified promastigotes lysate was incubated for 30±2 minutes using a water bath at 93±2° C. After heat treatment, the bulk was cooled at 4±2° C. Aseptically two 0.5 ml aliquots were taken and retained at −80±10° C. One 1.0 ml sample was taken for protein concentration testing by the Bio-Rad method (Bio-Rad Laboratories, Hercules, Calif.) and for SDS-PAGE.

The final clarified lysate protein concentration was aseptically adjusted to 0.35±0.05 mg/ml with 0.4% phenol buffer. Then samples were aseptically obtained and assayed for protein content by BCA, endotoxin content LAL (gel-clot), pH, sterility, HPLC, purity by SDS-PAGE, color and appearance.

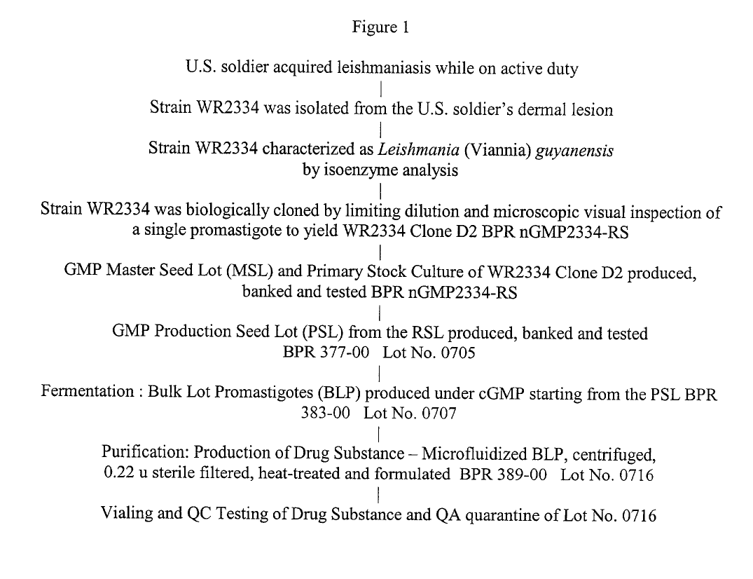

An Omnispense dispensing pump (Wheaton Science Products, Millvilee, N.J.) was used to dispense 1.0 g±5% of the final clarified lysate into 10 ml depyrogenized, sterile, glass vials. The bulk final clarified lysate was swirled on an orbital shaker at 60 rpm during the filling operation to ensure homogeneity. The final vials were inspected and weight checked. The vials were labeled and tested for sterility, pH, protein concentration, LAL, SDS-PAGE, HPLC and immunogenicity. FIG. 1 is a flow diagram of process for making the Leishmaniasis microfluidized lysate of the present invention.

### EXAMPLE 3

### Skin Test Antigen Assay in 10 Subjects

A microfluidized lysate preparation of L. tropica prepared according the Example 2 was tested in 10 Leishmania naïve human subjects. The microfluidized lysate preparation further included Tween-80® and dextran. The subjects received four doses two weeks apart of 0.1 ml of the microfluidized lysate preparation in escalating concentrations. The first dose had 0.25 μg, the second dose had 2.5 μg, the third dose had 8.0 μg, and the fourth dose had 25.0 μg of total protein. 25.0 μg of the preparation was found to be safe in six of the 10 subjects. One subject developed clear rhinorrhea and nasal pruritus within minutes of receiving the first dose. Four hours later, transient urticaria at the test and control-diluent sites occurred. This reaction was consistent with systemic manifestations of type I hypersensitivity to the dextran and diluent control. Three additional subjects also withdrew from the study after developing induration at the site of administration. The remaining subjects, except one subject who withdrew from the study due to a move out of the area, received three additional 25.0 μg doses of the microfluidized lysate to evaluate sensitization. The subject who moved from the area received only two additional 25.0 μg doses without difficulty.

As it was believed that the dextran caused the hypersensitivity in the subjects, the microfluidized lysate was reformulated. The reformulated microfluidized lysate preparation of L. tropica comprising 0.4% Phenol as a preservative was tested in 15 Leishmania naïve human subjects and was found to be safe as a single injection at doses of 0.38 μg, 3.8 μg, and 38.0 μg.

### EXAMPLE 4

### Heat-Treated Leishmania Skin Test Injectable Study

60 active leishmania subjects, 60 healthy leishmania subjects, and 60 healed leishmania subjects ages 18-55 are recruited to evaluate the safety of the microfluidized lysate preparation, determine the specificity and sensitivity of varying antigen doses, evaluate the sensitizing capacity of the microfluidized lysate preparation, and to compare the intensity of the induration responses evoked (cross-reactivity) when heterologous and homologous antigens are used.

A subject is deemed to be clinically diagnosed with an active Leishmaniasis infection upon the demonstration of motile promastigotes in aspirate cultures or microscopic Leishmania amastigotes in samples obtained from lesions obtained from the subject. The parasite may be visualized by conventional methods. A subject is deemed to be clinically healed upon 100% reepitheliazation of the ulcer.

All subjects are tested with an anergy panel comprising Mumps Skin Test Antigen, 40 CFU/ml, MSTA®, Connaught Labs Inc. (Swiftwater, PM), and Candida albicans skin test antigen, Candin®, Allermed Laboratories, Inc. (San Diego, Calif.), before receiving the preparations. Subjects who have a positive response to at least one of the antigens of the anergy panel, an induration of greater than about 5 mm, may participate.

Subjects who have a history of atopy, allergic reactions, or asthma will be excluded from the study including those who are allergic to phenol, pharmaceutical detergents or glycol. Also excluded are those subjects taking steroids, antihistamines, cimetidine, and immunosupressants, as well as those who had a splenectomy or have an active medical or psychiatric conditions that may increase the risks associated with participation in the study or interfere with the interpretation of study results. Pregnant or nursing subjects will be excluded. Subjects having active cutaneous Leishmaniasis with scars, i.e. possible re-infections and residivant Leishmaniasis will be excluded. Subjects having been immunized within 4 weeks prior to the start of the study will be excluded as well as those having anergy on delayed type hypersensitivity testing (less than about 5 mm of induration).

Primary endpoints are the occurrence of local or systemic reactions to the skin or the occurrence of non-specific immune responses to the skin tests. The microfluidized lysate preparations are considered safe if there are no clinically significant local or systemic reactions in the healthy and healed subjects. The microfluizided lysate preparations will also be considered safe if no severe reactions are determined in active leishmania subjects. Secondary endpoints are the size of the induration which accompanies the delayed type hypersensitivity response (potency); the percentage of subjects with Leishmaniasis in which the preparation induced a positive response about 5 mm or greater delayed type hypersensitivity reaction (sensitivity), the percent of healthy subjects in which the preparation fails to induce a positive delayed type hypersensitivity reaction (specificity).

The microfluidized lysate preparations are first tested in healthy subjects, followed by healed subjects, and followed by visceral or cutaneous subjects. In order to determine the magnitude of cross-reactivity of the preparations, the same subject is administered three different preparations, L. mexicana, L. tropica, and L. guyanensis, at a given concentration simultaneously. This study allows the direct comparison of the three preparations and reduces the number of subjects in the study. The study is a dose escalation study which tests three dose levels of the microfluidized lysate preparations. Three cohorts of 20 subjects per clinical group are used. The dose of each preparation escalates for each cohort. Intradermal injections of saline and a 1:100 dilution of the diluent are administered as controls concomitantly with the preparations. Initially, 20 healthy subjects (Cohort 1) are administered microfluidized lysate preparations having total protein concentrations of 5 μg (0.1 ml ID). If no clinically significant reaction is observed within two days after administration in Cohort 1, Cohort 2 is administered preparations having 15.0 μg total protein (0.1 ml ID). If no significant reaction is observed within two days after administration in Cohort 2, Cohort 3 is administered preparations having 30.0 μg total protein (0.1 ml ID). Each subject is followed for two days to assess local and systemic reactions.

Subjects who were tested more than once during previous leishmanin standardization studies or healthy subjects receiving one administration of the lysate preparation serve to evaluate whether repeated skin-testing is sensitizing (n=60). This “Sensitizing Group” will receive a second dose of the lysate preparations and will be tested last.

The controls are 0.1 ml saline and a 1:100 dilution of the microfluidized lysate preparation diluent comprising 0.001% Tween-80, 1% glycerol, 0.4% phenol diluted with 0.9% saline stored at 4±2° C. 0.1 ml of a microfluidized lysate preparation is injected intradermally into alcohol-cleansed volar surface of the forearm under the supervision of a physician who will have medication and equipment to treat anaphylaxis. The diameter of the induration, erythema, or both will be measured in millimeters at about 30 minutes after administration to detect immediate IgE hypersensitivity, and about 48 hours later to detect delayed type hypersensitivity by outlining the indurated border with a ballpoint pen and transferring to paper damped with 70% ethanol for permanent record. The largest diameter and its perpendicular diameter are measured and averaged. Alternatively, the Sokal method may be used. See Montenegro, J. (1926) Archives of Dermatology and Syphilology 13:187-194. The placement of each preparation and control for each subject are randomized and recorded. Skin test readers are unaware of the placements. Subjects are monitored closely.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

We claim:

1. A method of preparing a microfluidized lysate preparation comprising microfluidizing a slurry of at least one Leishmania parasite through a chamber and disrupting the leishmania parasite with a sudden release of pressure.

2. The method of claim 1, further comprising heat treating the microfluidized lysate preparation.

3. The method of claim 1, wherein the Leishmania parasite is L. tropica, L. mexicana, L. guyanensis, L. braziliensis, L. major, L. donovani, L. chagasi, L. amazonensis, L. peruviana, L. panamensis, L. pifanoi, L. infantum, or L. aethiopica.

4. A microfluidized lysate preparation made by the method of claim 1.

5. A skin test antigen assay for detecting whether a subject had been exposed to a Leishmania parasite or was afflicted with Leishmaniasis comprising administering to the subject an antigenic amount of at least one microfluidized lysate preparation according to claim 4 and observing any immunogenic response to the microfluidized lysate preparation.

6. The skin test antigen assay of claim 5, wherein the Leishmania parasite is L. tropica, L. mexicana, L. guyanensis, L. braziliensis, L. major, L. donovani, L. chagasi, L. amazonensis, L. peruviana, L. panamensis, L. pifanoi, L. infantum, or L. aethiopica.

7. The skin test antigen assay of claim 5, wherein an immunogenic response indicates that the subject had been exposed to a Leishmania parasite or was afflicted with Leishmaniasis.

8. The skin test antigen assay of claim 5, wherein an induration of about 5 mm or greater observed indicates that the subject had been exposed to a Leishmania parasite or was afflicted with Leishmaniasis.

9. The skin test antigen assay of claim 5, wherein the antigenic amount of the microfluidized lysate preparation comprises about 5 μg to about 30 μg of total protein.

10. The skin test antigen assay of claim 5, wherein the antigenic amount of the microfluidized lysate preparation is administered intradermally to the volar surface of the forearm of the subject.

11. A kit comprising the microfluidized lysate preparation of claim 4 and directions for determining whether a subject has been exposed to a Leishmania parasite or was afflicted with Leishmaniasis.

12. The kit of claim 11, wherein the Leishmania parasite is L. tropica, L. mexicana, L. guyanensis, L. braziliensis, L. major, L. donovani, L. chagasi, L. amazonensis, L. peruviana, L. panamensis, L. pifanoi, L. infantum, or L. aethiopica.

13. The kit of claim 11, further comprising at least one pharmaceutical for treating systemic anaphylaxis.

14. The kit of claim 13, wherein the pharmaceutical is epinephrine, diphenhydramine, or methyl prednisolone.

15. The kit of claim 11, further comprising at least one pharmaceutical for treating local reactions to the microfluidized lysate preparation.

16. The kit of claim 15, wherein the pharmaceutical is hydrocortisone, hydrocortisone cream, acetaminophen, or diphenhydramine.

17. An antibody raised against the microfluidized lysate preparation of claim 4.

18. A vaccine comprising the microfluidized lysate preparation of claim 4.

19. A method of determining whether a subject has been exposed to a given Leishmania parasite comprising administering to the subject a panel of antigenic compositions comprising a plurality of microfluidized lysate preparations prepared from a plurality of Leishmania parasites and detecting a presence of an immunogenic reaction that is characteristic to exposure to the given Leishmania parasite.

20. The method of claim 19, wherein the plurality of Leishmania parasites comprises at least one parasite from the group consisting of L. tropica, L. mexicana, L. guyanensis, L. braziliensis, L. major, L. donovani, L. chagasi, L. amazonensis, L. peruviana, L. panamensis, L. pifanoi, L. infantum, and L. aethiopica.

21. A method of immunizing a subject against Leishmaniasis comprising administering to the subject an immunogenic amount of the microfluidized lysate preparation of claim 4.

22. A pharmaceutical composition comprising the microfluidized lysate preparation of claim 4 and a pharmaceutically acceptable stabilizer.

23. The pharmaceutical composition of claim 22, wherein the pharmaceutically acceptable stabilizer is phenol.

24. The pharmaceutical composition of claim 22, wherein the composition is in the form of a liquid.

25. The pharmaceutical composition of claim 22, wherein the composition may be frozen or freeze-dried.

26. A method for determining post infection of cutaneous leishmaniasis, mucocutaneous leishmaniasis, or post-kala-azar dermal leishmaniasis in a subject comprising administering to the subject an antigenic amount of at least one microfluidized lysate preparation of claim 4 and observing any immunogenic response to the microfluidized lysate preparation.

27. A method for epidemiologically diagnosing cutaneous leishmaniasis, mucocutaneous leishmaniasis, or post-kala-azar dermal leishmaniasis in a subject comprising administering to the subject an antigenic amount of at least one microfluidized lysate preparation of claim 4 and observing any immunogenic response to the microfluidized lysate preparation.

28. A method for determining the pattern of present and past leishmaniasis in a subject comprising administering to the subject an antigenic amount of at least one microfluidized lysate preparation of claim 4 and observing any immunogenic response to the microfluidized lysate preparation.
